(19) United States
(12) Patent Application Publication
(10) Pub. No.: US 2023/0263547 A1
(43) Pub. Date: Aug. 24, 2023

(54) HYDRO DISSECTION AND SUCTION LAPAROSCOPIC FORCEPS AND METHODS OF USE

(71) Applicants: Dinesh Vyas, Elk Grove, CA (US); Albert K. Chin, Palo Alto, CA (US)

(72) Inventors: Dinesh Vyas, Elk Grove, CA (US); Albert K. Chin, Palo Alto, CA (US)

(21) Appl. No.: 18/089,368

(22) Filed: Dec. 27, 2022

Related U.S. Application Data
(60) Provisional application No. 63/312,770, filed on Feb. 22, 2022.
(60) Provisional application No. 63/421,511, filed on Nov. 1, 2022.

(51) Int. Cl. A61B 17/295; A61B 17/3203; A61B 18/14

(52) U.S. Cl. CPC A61B 17/295; A61B 17/3203; A61B 18/1445; A61B 2018/00595

(57) ABSTRACT

A laparoscopic instrument includes fluid carrying channels incorporated into lateral aspects of jaws of the instrument and extending to outlets adjacent distal tips of the jaws, fluid carrying channels incorporated into a shaft of the instrument, and flexible fluid carrying channels connecting the distal ends of the shaft channels with the proximal end of respective jaw channels. An actuator on the device handle controls either delivery of fluid jets emanating lateral to tissue grasped between the instrument jaws or suction from the distal tips of the jaws to remove fluid in the vicinity of the jaws. The fluid jets perform atraumatic tissue dissection lateral to the structures grasped by the jaws, while the grasping jaws provide counter traction while conducting soft tissue hydro-dissection, by stabilizing the tissue in a direction opposite to the force exerted by the hydro-dissection fluid jets.

### RELATED APPLICATION DATA

The present application claims benefit of co-pending U.S. provisional applications Ser. No. 63/312,770, filed Feb. 22, 2022, and 63/421,511, filed Nov. 1, 2022, the entire disclosures of which are expressly incorporated by reference herein.

### TECHNICAL FIELD

The present application relates to devices and methods for tissue dissection, e.g., conducted in laparoscopic surgery, which may involve blunt tissue dissection performed via a focused fluid jet instead of the mechanical tissue disruption typically performed by laparoscopic dissectors and graspers. In one example, the instrument is a laparoscopic grasper with double action jaws that include rigid nozzles on lateral aspects of each jaw. Rigid fluid carrying channels extend along lateral aspects of the instrument shaft, and a short length of flexible hose connects the distal end of each channel to a proximal end of a respective jaw nozzle channel. The short flexible hose sections may adopt a substantially straightened position when the jaws are closed, such that their outer profile does not exceed the outer profile of the instrument shaft, allowing the instrument to be inserted through a trocar, e.g., a five millimeter (5 mm) laparoscopic trocar.

### BACKGROUND

In laparoscopic surgical procedures, isolation of anatomic structures, such as blood vessels and ducts, are performed via blunt dissection maneuvers involving spreading and tearing of soft tissue adjacent to the vessels and ducts. If the organ involved in the surgery is ischemic or necrotic, the organ and surrounding soft tissue becomes swollen and edematous, making it impossible to discern the outlines and locations of underlying ducts and vessels. Surgical maneuvers with existing laparoscopic graspers and dissectors carry significant potential of disrupting unrecognized organs, ducts, and vessels, which may lead to spillage of toxic infective contents into the abdominal cavity and hemorrhage.

Blunt dissection of soft tissue during laparoscopic surgery may be hazardous when the tissue is swollen and edematous, and the outline of blood vessels and ducts coursing through the soft tissue is not visible via endoscopic observation. In gangrenous cholecystitis, the gallbladder is distended to such a degree that it becomes ischemic, with compromise to its blood supply. Necrosis of the organ occurs, and the resultant inflammation and tissue swelling in the area of the gallbladder obscures the location of the cystic duct and the cystic artery. These structures must be surgically isolated, ligated or clipped, and transected for gallbladder removal. Mechanical dissection of the gangrenous gallbladder with traditional laparoscopic instruments such as Maryland dissectors may easily cause transection or laceration of non-visualized cystic duct and cystic artery, causing spillage of infected bile in the abdominal cavity and hemorrhage.

Other endoscopic procedures requiring execution of difficult and hazardous surgical blunt dissection include intra-abdominal endometriosis lesion resection, adhesiolysis or lysis of adhesions, and video assisted thoracic surgery or VATS, generally involving lung resection procedures and drainage of empyema.

Previous laparoscopic forceps exist that supply fluid irrigation and suction to the jaws of the instruments. One such instrument, described by Fischer in U.S. Pat. No. 9,308,014, teaches the use of a fluid jet at or in a stationary jaw of a forceps to dissect tissue.

Therefore, improved devices and methods for performing dissection of tissue within a patient's body would be useful.

### SUMMARY

The present application relates generally to surgical devices and, more particularly, to devices for tissue dissection, e.g., conducted in laparoscopic surgery, and to methods for making and using such devices.

In one example, a hydro dissection and suction laparoscopic forceps device is provided that includes two movable jaws connected to distal end of a long rigid, e.g., five millimeter (5 mm) outer diameter, shaft, with the jaws configured to be opened and closed by a handle on a proximal end of the shaft, e.g., including an elongated stationary ring that accommodates multiple fingers and a movable thumb ring that actuates the jaws. Rigid tubular channels may be provided on lateral aspects of the shaft and jaws, e.g., including a pair of shaft channels extending between the proximal and distal ends of the shaft and a relatively short, e.g., three millimeter (3 mm) long, tubular jaw channel on each of the jaws, which are circumferentially exposed to allow attachment of two flexible tubes connecting each shaft channel to a corresponding jaw channel in a fluid tight fashion. The flexible tubes may allow the closed jaw instrument to maintain a desired maximum outer diameter or other profile, e.g., a five millimeter (5 mm) outer diameter throughout the length of the device, e.g., for insertion through a corresponding, e.g., five millimeter (5 mm), inner diameter trocar.

Once the jaws are disposed inside an abdominal cavity of a patient, the jaws may be opened and closed while the flexible tubes allow fluid delivery lateral to the jaws for atraumatic dissection of tissue lateral to anatomic structures grasped by the jaws, e.g., as counter traction is applied to the tissue stabilized by the forceps. Such a method of lateral tissue hydro dissection while applying centralized tissue counter traction may be less traumatic to tissue than conventional blunt dissection with conventional laparoscopic forceps, as the such conventional forceps typically involve tissue puncture and tissue tearing as components of mechanical blunt dissection. In contrast, hydro dissection, as enabled by the devices and methods herein, uses gentle fluid jet streams to separate tissue and isolate anatomic structures, eliminating the sharp force tissue interaction associated with mechanical blunt dissection.

In laparoscopic procedures, it is desirable to perform surgery with forceps containing double action jaws rather than a single action jaw and a stationary jaw. Double action jaws permit a wider grasp of tissue to prevent slippage during tissue manipulation. Double action jaws also enable the jaws to remain in axial orientation with the shaft of the instrument, with the jaws opening symmetrically on either side of the shaft. Application of the laparoscopic forceps with double action jaws is more intuitive, facilitating efficient surgical technique and saving operative time. In contrast, with a single action forceps, the instrument shaft needs to be displaced to the side of the stationary jaw to accurately grasp tissue as intended. Thus, double action jaws on the device may be particularly useful, although, alternatively, single action jaws may also be provided, if desired.

In one example, the hydro dissection and suction laparoscopic forceps devices provided herein may incorporate a self-contained fluid pump and/or a battery or other power source to power the pump, e.g., within the handle device. Saline may be supplied to the pump via an intravenous line, e.g., attached to a hanging intravenous fluid bag. Optionally, a separate hose supplying wall suction in the operating room is also connected to the device handle.

In one example, the device may include two normally-closed trumpet valves, e.g., which reside in series in the intravenous line attached to both tubular channels on the proximal end of the shaft. For example, one trumpet valve may control both the electrical supply to the fluid pump and the fluid flow lateral to the forceps jaws, and the other trumpet valve may activate vacuum to clear fluid injected during hydro dissection.

Optionally, the laparoscopic hydro dissection forceps devices herein, e.g., including the fluid pump and the battery, may be a single-use device that is disposed following the surgical procedure, to avoid the need for device cleaning, re-sterilization and storage between successive procedures. Alternatively, all or some components of the device may be reusable, e.g., after cleaning and/or sterilization.

In one example, the fluid carrying tubular channels located on the grasper jaws may have a smaller inner diameter than the tubular channels located on the lateral aspects of the instrument shaft. This allows the velocity of the fluid jet emanating from the grasper jaw channels to be tuned to a desired level, e.g., based on pressure and/or flow rate specifications generated by the fluid pump.

Pathologic conditions may exist that cause anatomic landmarks to be obscured during endoscopic surgery, rendering tissue dissection difficult and hazardous. For example, in laparoscopic cholecystectomy or removal of the gallbladder, surgical dissection must be performed to isolate the cystic artery and cystic duct to allow for their ligation and transection prior to gallbladder removal. Acute cholecystitis is inflammation of the gallbladder caused by occlusion of the cystic duct by gallstones. The gallbladder becomes distended, and the pressure inside the organ may increase to such a level that it compromises the blood supply and causes ischemia, leading to gangrenous cholecystitis, which occurs in over 20% of acute cholecystitis cases. The severe inflammation observed in gangrenous cholecystitis causes such a degree of swelling and edema in the gallbladder and surrounding tissues that the outlines of anatomic structures, such as the cystic duct and cystic artery are invisible under laparoscopic visualization, and normally observed outlines and landmarks are obscured.

Blunt tissue dissection using conventional laparoscopic forceps requires insertion of the closed tips of the forceps jaws into tissue without perceptible landmarks, followed by opening of the jaws to spread apart the tissue. The tissue disruption associated with this blunt dissection maneuver may easily lacerate or transect unseen vessels and ducts. In contrast, hydro dissection is a less traumatic approach to isolation of anatomic structures embedded in edematous tissue. Surgical dissection of inflamed tissue also prolongs procedure times, increasing the physical stress of surgery and general anesthesia to the patient, thus increasing the patient morbidity and mortality.

A modified technique of tissue dissection is proposed herein, involving tissue dissection performed solely by simultaneous hydro-dissection lateral to both sides of double action movable laparoscopic grasper jaws. The jaws of the devices described herein may not be applied in a typical fashion for mechanical tissue disruption and blunt dissection. Rather, the jaws may gently grasp and fixate exposed tissue prior to instillation of pressurized fluid jets lateral to the grasping jaws to perform hydro dissection of the soft tissue to achieve isolation of the desired anatomic structures.

The hydro dissection devices and methods herein may also be used in additional endoscopic procedures, e.g., to isolate delicate anatomic structures obscured by overlying amorphous tissue, such as resection of intra-abdominal endometriosis lesions, lysis of tissue and organ adhesions, and video assisted thoracic surgical procedures such as lung resection and lobectomy. In these procedures, dissection of connective tissue surrounding delicate organs, blood vessels and ducts may be performed less traumatically via hydro dissection versus standard mechanical blunt surgical dissection.

In accordance with one example, a device is provide for performing hydro-dissection of tissue within a patient's body that includes an elongate shaft comprising a proximal end, a distal end sized for introduction into the patient's body, and one or more shaft channels extending between the proximal and distal ends; first and second jaws on the distal end coupled to an actuator on the proximal end for moving the jaws between closed and opened positions, each jaw comprising a jaw channel comprising an outlet disposed adjacent a distal tip of the respective jaw; and a flexible tube extending between each jaw and the distal end of the shaft to fluidly couple the outlet of the respective jaw to the one or more shaft channels to deliver pressurized fluid from a fluid source through the one or more shaft channels, the flexible tubes, the jaw channels, and out the outlets to dissect tissue adjacent grasped between the jaws.

In accordance with another example, a device is provided for performing hydro-dissection of tissue within a patient's body that includes an elongate shaft comprising a proximal end, a distal end sized for introduction into the patient's body, and first and second shaft channels extending between the proximal and distal ends; first and second jaws on the distal end, each jaw comprising an outlet disposed adjacent a distal tip of the respective jaw; an actuator on the proximal end coupled to the jaws to manipulate the jaws between closed and opened positions; first and second flexible tubes extending between the jaws and the distal end of the shaft communicating between the outlet of the respective jaws and the first and second shaft channels, respectively; and a source of pressurized fluid coupled to the first and second shaft channels to deliver pressurized fluid through the shaft channels, the flexible tubes, the jaw channels, and out the outlets to dissect tissue adjacent grasped between the jaws.

In accordance with still another example, a method is provided for dissecting tissue within a patient's body that includes providing a dissection device comprising a distal end carrying a pair of jaws, each jaw comprising a nozzle adjacent a distal tip of the jaw; introducing the distal end into the patient's body with the jaws in a closed position; opening the jaws; manipulating the device and jaws to grasp tissue between the jaws; and delivering pressurized fluid out the nozzles to dissect tissue adjacent the jaws.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures.

FIGS. 1a and 1b depict the appearance of an exemplary non-gangrenous and gangrenous gallbladder, respectively.

FIGS. 2a and 2b show exemplary maneuvers used in mechanical blunt dissection of tissue.

FIGS. 3a-3c depict an exemplary sequence of steps that may be used in hydro dissection of tissue, e.g., using the hydro-dissection devices described herein.

FIG. 4 illustrates exemplary differences in width of jaw opening between a single action jaw instrument and a double action jaw instrument.

FIG. 5 depicts exemplary components of a hydro dissection laparoscopic forceps device.

FIGS. 6a-6c illustrate details of a distal portion of the device of FIG. 5.

FIGS. 7a and 7b depict an exemplary profile of the jaws of the device of FIG. 5 in closed and open configurations, respectively.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

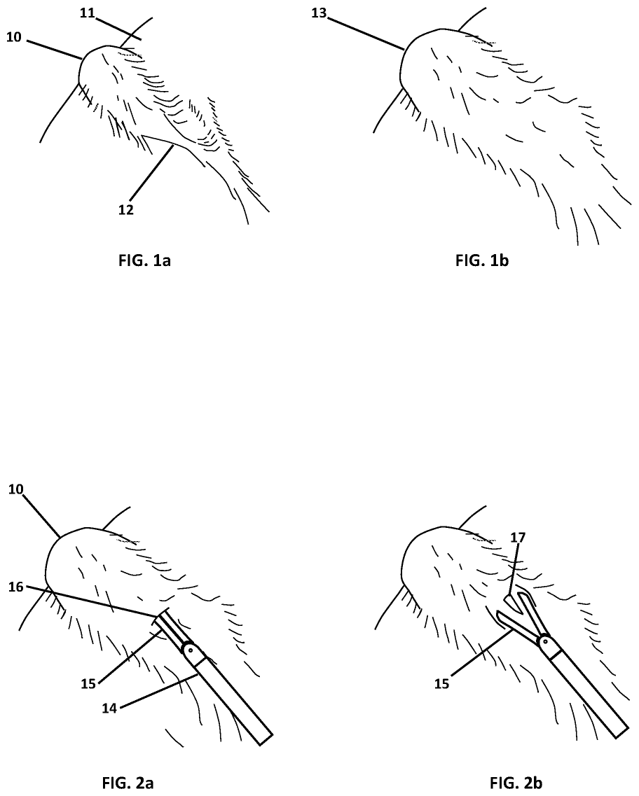

Turning to the drawings, FIG. 1a illustrates an example of the surgical appearance of a gallbladder 10 attached to the underside of a liver 11, in a non-gangrenous situation. The outline of the cystic duct 12 may typically be observed in a non-gangrenous laparoscopic cholecystectomy surgery. FIG. 1B depicts an example of a gangrenous gallbladder 13, that is enlarged, swollen and edematous. The outline of the gangrenous gallbladder 13 and normal surrounding anatomical landmarks are obscured by the inflamed and edematous tissue.

FIG. 2a depicts an example of mechanical blunt dissection of the gallbladder 10 using conventional laparoscopic forceps 14. The tips of the jaws 15 of the laparoscopic forceps 14 are inserted into tissue at an entry site 16, with the jaws 15 in a closed orientation. Following tissue insertion, the jaws 15 are forcibly opened, as shown in FIG. 2b, to dissect the tissue of gallbladder 10, creating a cleavage plane or opening 17 in the bluntly dissected tissue. Blunt mechanical dissection of tissue is generally safe to perform when the gallbladder is non-gangrenous, and underlying anatomical structures such as blood vessels and ducts are visually perceived. If underlying blood vessels and ducts are obscured, however, e.g., due to tissue edema and inflammation in the event of gangrenous cholecystitis and the like, laceration, perforation, and/or transection of blood vessels and ducts may occur upon blunt dissection maneuvers using conventional laparoscopic forceps. Application of a fluid jet to dissect tissue in gangrenous gallbladder may be useful, as a moderately pressurized fluid jet imparts less force than rigid forceps jaws during tissue dissection, particularly in inflamed and edematous tissue that is more friable than normal tissue.

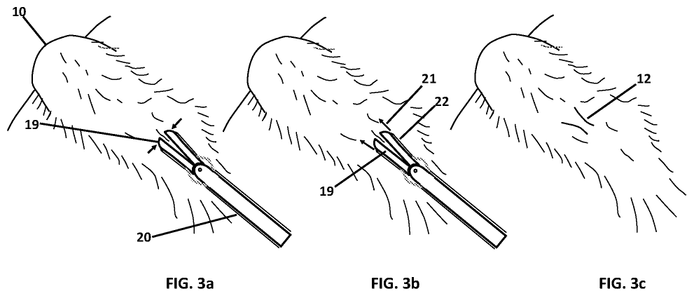

FIG. 3a-3c illustrate an exemplary technique of atraumatic tissue dissection using a hydro dissection laparoscopic forceps device 20. In FIG. 3a, the jaws 19 of the hydro dissection forceps 20 gently close on tissue of the gallbladder 10 without its tips puncturing into the tissue. Following closure of the jaws 19, the hydro dissection forceps device 20 may remain in a fixed position, i.e., the device 20 is not moved to tear or otherwise displace tissue. In FIG. 3b, following fixation of the tissue by closure of the jaws 19, fluid jets 21 are initiated, e.g., lateral to each jaw 19. The closed jaws 19 fixate tissue and provide counter traction as the fluid jets 21 exert hydro dissection force against tissue lateral to the region grasped by the jaws 19. Tissue separation is performed solely by hydro dissection, and avoidance of conventional blunt dissection maneuvers involving tissue tearing and tissue puncture leads to atraumatic isolation of anatomic structures, such as the duct 12 observed in FIG. 3c.

FIG. 4 illustrates functional differences that may be experienced between forceps 23 with a single action jaw 24 and double action forceps 20 with two moveable jaws 19. Jaws 19 of double action forceps 20 open twice as wide as forceps 23 with a single moveable jaw 24, permitting a surgeon to grasp a larger amount of tissue between the jaws 19 for enhanced tissue control and avoidance of tissue slippage out of the grasp of jaws 19. Proper tissue fixation is necessary to provide requisite counter traction during lateral hydro dissection procedures, particularly with the wet tissue environment encountered with the technique. Thus, double action, movable jaws 19 may provide advantages over a single action jaw 24 during tissue dissection.

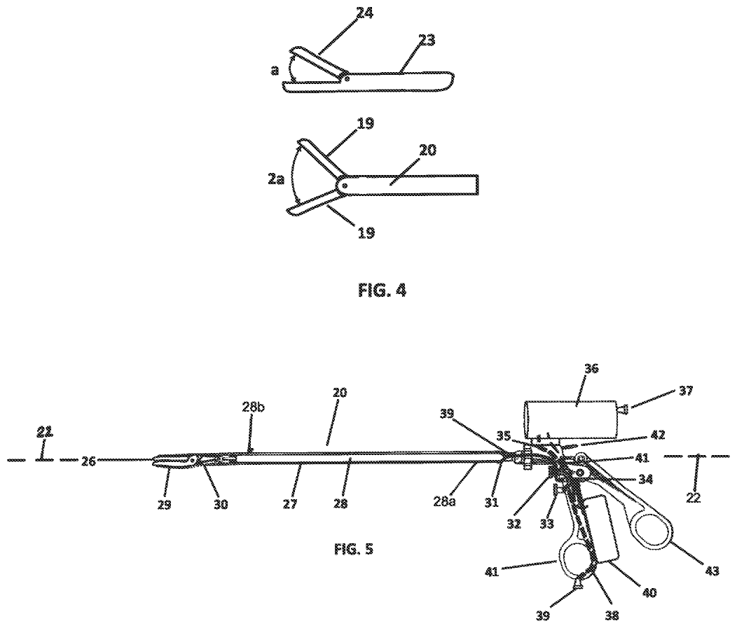

Turning to FIG. 5, exemplary components are shown that may be included in a hydro dissection laparoscopic graspers device 20. Generally, the device 20 includes an elongate shaft 28 defining a longitudinal axis 22, e.g., a substantially rigid tubular or solid shaft, including a proximal end 28a and a distal end 28b sized for introduction into a patient's body. The shaft 28 may include a pair of shaft channels 27, e.g., including diametrically opposed rigid fluid supply tubes 27 attached to lateral aspects of the shaft 28. Alternatively, the shaft channels 27 may be integrally formed in the wall of the shaft 28, e.g., by one or more of extrusion, molding, casting, machining, and the like. In a further alternative, the shaft 28 may include a single channel or infusion lumen extending from the proximal end 28a to the distal end 28b and a fitting split, or other branch (not shown) may be provided on the distal end to provide two openings that may be connected to respective flexible tubes and jaw channels 29.

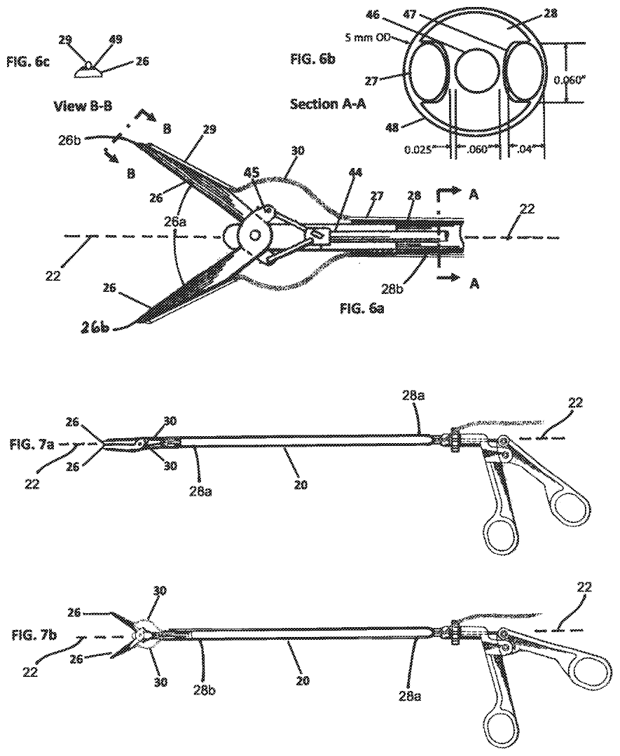

The device 20 includes a pair of jaws 26 on the distal end 28b of the shaft 28, e.g., that may be manipulated between a closed position (e.g., as shown in FIG. 7a) and an open position (e.g., as shown in FIG. 7b), via manipulation of an actuator 43 on a handle 41 on the proximal end 28a of the shaft 28. In the example shown, the handle 41 includes a stationary ring extension 41 and a moveable ring actuator 43 coupled to the jaws 26 to direct them between the closed and open positions. As best seen in FIG. 6a, the jaws 26 are coupled to the distal end 28b of the shaft 28 such that, manipulation of the actuator 43 causes both jaws 26 to move laterally away from the axis 22 when the jaws 26 are opened and the jaws 26 are generally aligned along the axis 22 when the jaws are closed, e.g., as shown in FIGS. 5 and 7a. Alternatively, the device may include only a single movable jaw and a fixed jaw attached to or otherwise extending from the distal end of the shaft (not shown), e.g., similar to the forceps 23 shown in FIG. 4. In this alternative, an actuator may move the single jaw between open and closed positions to grasp tissue between the jaws.

Optionally, the jaws 26 may include substantially blunt surfaces to prevent puncturing, cutting, and/or otherwise damaging tissue. For example, as shown, the inner contact surfaces 26a and/or the distal tips 26b of the jaws 26 may include flat and/or rounded edges to allow tissue to be grasped between the jaws 26 with minimal risk of tearing or cutting.

As best seen in FIG. 6a, fluid supply tubes 29 may be attached and/or otherwise provided on the jaws 26, e.g., extending along lateral aspects of the jaws 26 on opposite outer longitudinal edges. For example, separate substantially rigid tubular segments may be formed and permanently attached to the outer edges of the jaws 26, e.g., by one or more of bonding with adhesive, welding, soldering, brazing, fusing, and the like, or, alternatively, the jaw tubes 29 may be integrally formed with the jaws 26. A flexible tube 30 extends from each shaft channel 27 to a respective jaw tube 29, e.g., attaching the distal end of shaft fluid supply tubes 27 to jaw fluid supply tubes 29 in a fluid tight manner. The flexible tubes 30 allow fluid delivery while the jaws 26 are opened, closed, or partially open.

In one example, when the jaws 26 are closed, the outer profile of the working portion of the device 20, including the jaws 26, fluid supply channels 29 and 27, and flexible tubes 30 do not exceed about five millimeters (5 mm) or other maximum outer diameter or cross-section, e.g., to allow the device 20 to be introduced into the body through a corresponding sized access device, e.g., a five millimeter (5 mm) laparoscopic or thoracoscopic trocar (not shown).

Optionally, the graspers 20 may be connected to a radiofrequency power source (not shown), e.g., via a connector 42 on the proximal end 28a, e.g., on handle 41 as shown in FIG. 5, to allow the jaws 26 to cauterize blood vessels, ducts, and/or other tissue, similar to conventional laparoscopic graspers. The connector 42 may be coupled to the jaws 26 by one or wires or other leads (not shown) extending between the proximal and distal ends 28a, 28b of the shaft 28, which may, in turn, to electrically conductive electrodes or surfaces on the jaws 26. For example, the entire inner contact surface 26a of the jaws may be coupled to the leads to deliver electrical energy to tissue contacted between the jaws 26, if desired.

Pressurized fluid for hydro dissection is supplied by a miniature fluid pump 36 included in or coupled to the handle 41, e.g., integrated into the superior aspect of the stationary portion of the handle 41. The fluid pump 36 may include a connector, e.g., a female luer fitting 37, that accepts an intravenous fluid line connected to a saline bag or other source of fluid (not shown), e.g., containing one to three (1-3) liters or other desired volume of sterile saline. In one example, a fluid supply line 35 extends from the pump 36 to a normally closed fluid irrigation trumpet valve 32 provided on the handle 41.

Optionally, a connector 39 may be provided to connect a source of vacuum or suction (not shown) to the device 20, e.g., communicating with the shaft channels 27. For example, as shown in FIG. 5, the stationary handle portion 41 may include a suction luer fitting 39 that allows the hydro dissection laparoscopic graspers 20 to be connected to wall suction in the operating or procedure room, for evacuation of fluid via jaw channels 29. As shown, a suction connecting tube 38 extends from luer fitting 39 to the inlet of a suction trumpet valve 33, which is also in a normally closed position until the trumpet valve 33 is depressed.

In one example, the outlets of both irrigation trumpet valve 32 and suction trumpet valve 33 are connected together to a common fitting 34 with a connecting line 39 extending to a connector 31 that is attached to both fluid supply tubes 27. Thus, in this example, depression of the irrigation trumpet valve 32 causes fluid dissection jets to emanate from both fluid supply tubes 27, while depression of the suction trumpet valve 33 causes suction of fluid via the shaft and jaw channels 27, 29. If the jaw and/or shaft channels 29, 27 become clogged during actuation in the suction mode, the irrigation mode may be activated to clear debris lodged in channels 29, 27.

FIG. 6a depicts an enlarged view of the jaws 26 and the distal end 28b of the shaft 28 of the device 20. Each jaw channel 29 is attached to or otherwise extends from the lateral aspect of jaw 26, e.g., along the outer edge of each jaw 29. A proximal end of each jaw channel 29 may be offset distally from the hinge connecting the jaws 29, e.g. offset about three millimeters (3 mm) from the proximal end of each jaw 26, e.g., to provide circumferential clearance to permit attachment of the flexible tube 30 and/or accommodate rotational movement of the jaws 26. Similarly, each shaft channel 27 is attached to or otherwise extends axially along either side of the shaft 28, except for desired offset, e.g., about a three millimeters (3 mm) long length of its distal portion, where circumferential clearance permits attachment of the proximal end of flexible tube 30.

An actuator may be coupled to the jaws 26 to manipulate the jaws between the open and closed positions. For example, as shown, the jaws 26 are opened and closed via advancement and retraction using an elongate member, e.g., a stainless steel rod 44 that extends through the length of shaft 28 and connects to jaw actuation linkage 45. FIG. 6b is a cross-section of the shaft 28, showing a central channel 46 that accommodates actuation rod 44 in a sliding fashion within its lumen. In the example shown, two lateral grooves 47 may extend the length of shaft 28, to allow the attachment of fluid tubes 27, which may be elliptical rather than circular in cross-section, if desired, to increase its luminal area for maximal fluid delivery. Optionally, a relatively thin outer sheath 48, e.g., composed of polymer heat shrink or other material, may cover the shaft 28 and attached fluid tubes 27, to provide a substantially smooth outer surface for insertion and sealing in a laparoscopic trocar port, and/or to electrically insulate the outside of shaft 28 as radiofrequency current is applied to cauterize tissue grasped by jaws 26.

FIG. 6c is an end view of an exemplary instrument jaw 26, illustrating the attachment of a fluid tube 29 to its lateral aspect. The jaw 26 may be thinned out, or a groove may be cut along its length to allow attachment of fluid tube 29 while maintaining a desired outer profile, e.g., not more than about five millimeters (5 mm) when jaws 26 are closed. The fluid tube 29 may be attached to the lateral aspect of jaw 26 using one or more of adhesive, solder, braze, weld, and the like. The attachment material may form a contoured fillet 49 along the length of the attached tube 29, to yield a smooth profile of the lateral aspect of the jaw 26, for atraumatic tissue contact during surgery.

FIG. 7a depicts the hydro dissection laparoscopic forceps 20 with the jaws 26 in a closed position. In the example shown, the flexible fluid supply connecting tubes 30 remain substantially flush against the forceps 20 when jaws 26 are closed, allowing unimpeded insertion through a desired access device, e.g., a 5 mm endoscopic trocar (not shown). FIG. 7b shows forceps 20 with the jaws 26 in an open position. Flexible connecting tubes 30 splay out laterally when the jaws 26 are opened. Forceps 20 insertion and removal must be conducted only with closed instrument jaws 26.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. A device for performing hydro-dissection of tissue within a patient's body, comprising:
an elongate shaft comprising a proximal end, a distal end sized for introduction into the patient's body, and one or more shaft channels extending between the proximal and distal ends;
first and second jaws on the distal end coupled to an actuator on the proximal end for moving the jaws between closed and opened positions, each jaw comprising a jaw channel comprising an outlet disposed adjacent a distal tip of the respective jaw; and
a flexible tube extending between each jaw and the distal end of the shaft to fluidly couple the outlet of the respective jaw to the one or more shaft channels to deliver pressurized fluid from a fluid source through the one or more shaft channels, the flexible tubes, the jaw channels, and out the outlets to dissect tissue adjacent grasped between the jaws.

2. The device of claim 1, wherein the one or more shaft channels comprise first and second shaft channels extending between the proximal and distal ends of the shaft, the first shaft channel fluidly coupled to the jaw channel of the first jaw by a first flexible tube and the second shaft channel fluidly coupled to the jaw channel of the second jaw by a second flexible tube.

3. The device of claim 1, further comprising a source of pressurized fluid coupled to the proximal end of the shaft that is fluidly coupled to the one or more shaft channels.

4. The device of claim 3, wherein the source of pressurized fluid comprises a pump carried on the proximal end of the shaft.

5. The device of claim 3, wherein the source of pressurized fluid comprises a reservoir of fluid carried on the proximal end of the shaft.

6. The device of claim 3, wherein the pump comprises a connector for coupling the source of pressurized fluid to the pump.

7. The device of claim 3, further comprising a connector on the proximal end of the shaft configured to be coupled to a source of suction, the connector coupled to a valve communicating with the one or more shaft channels.

8. The device of claim 7, further comprising an actuator for selectively opening the valve to alternatively deliver fluid and suction via the one or more shaft channels.

9. The device of claim 1, further comprising one or more ports on the proximal end of the shaft communicating with the one or more shaft channels configured to be coupled to an external source of pressurized fluid.

10. The device of claim 1, wherein the jaws comprise substantially blunt contact surfaces configured to grasp tissue between the jaws in the closed position.

11. The device of claim 1, wherein the shaft defines a longitudinal axis extending between the proximal and distal ends and wherein the outlets of the jaws are oriented distally on the distal tips to direct pressurized fluid substantially along the longitudinal axis when the jaws are in the closed position.

12. The device of claim 1, wherein the jaw channels have an inner cross-section that is smaller than the shaft channels.

13. The device of claim 1, further comprising:
a connector on the proximal end connectable to a source of electrical power;
one or more leads coupled to the connector and extending between the proximal and distal ends; and
an electrically active component on one or both of the jaws coupled to the one or more leads.

14. The device of claim 13, further comprising a generator configured to be coupled to the connector to deliver electrical energy to the electrically active component via the one or more leads to cauterize tissue contacted by the jaws.

15. The device of claim 13, wherein the electrically active component comprises one of an electrode and an electrically conducive surface on one or both of the jaws.

16. A device for performing hydro-dissection of tissue within a patient's body, comprising:
an elongate shaft comprising a proximal end, a distal end sized for introduction into the patient's body, and first and second shaft channels extending between the proximal and distal ends;
first and second jaws on the distal end, each jaw comprising an outlet disposed adjacent a distal tip of the respective jaw;
an actuator on the proximal end coupled to the jaws to manipulate the jaws between closed and opened positions;
first and second flexible tubes extending between the jaws and the distal end of the shaft communicating between the outlet of the respective jaws and the first and second shaft channels, respectively; and
a source of pressurized fluid coupled to the first and second shaft channels to deliver pressurized fluid through the shaft channels, the flexible tubes, the jaw channels, and out the outlets to dissect tissue adjacent grasped between the jaws.

17. The device of claim 16, wherein the source of pressurized fluid comprises a pump carried on the proximal end of the shaft.

18. A method for dissecting tissue within a patient's body, comprising:
providing a dissection device comprising a distal end carrying a pair of jaws, each jaw comprising a nozzle adjacent a distal tip of the jaw;
introducing the distal end into the patient's body with the jaws in a closed position;
opening the jaws;
manipulating the device and jaws to grasp tissue between the jaws; and
delivering pressurized fluid out the nozzles to dissect tissue adjacent the jaws.

19. The method of claim 18, wherein the jaws grasp the tissue to provide counter-traction during delivery of the pressurized fluid.

20. The method of claim 18, further comprising one or both of:
delivering suction via the nozzle of one or both jaw to aspirate material adjacent the distal end; and
delivering energy to one or both jaws to cauterize tissue adjacent the jaws.
